(19) United States
(12) Patent Application Publication
(10) Pub. No.: US 2025/0261903 A1
(43) Pub. Date: Aug. 21, 2025

(54) S4 BASED THERAPY ADJUSTMENT IN HCM PATIENTS

(71) Applicant: Cardiac Pacemakers, Inc., St. Paul, MN (US)

(72) Inventors: Pramodsingh Hirasingh Thakur, Woodbury, MN (US); Viktoria A. Averina, Shoreview, MN (US); Mojgan Goftari, Shoreview, MN (US)

(21) Appl. No.: 19/056,147

(22) Filed: Feb. 18, 2025

Related U.S. Application Data
(60) Provisional application No. 63/555,636, filed on Feb. 20, 2024.
(60) Provisional application No. 63/565,440, filed on Mar. 14, 2024.

(51) Int. Cl. A61B 5/00; A61B 5/0205; A61B 5/0537; A61B 5/08; A61B 7/04; G16H 20/10; G16H 20/30; G16H 20/40; G16H 50/20

(52) U.S. Cl. CPC A61B 5/4848; A61B 5/0205; A61B 7/04; G16H 20/10; G16H 20/30; G16H 20/40; G16H 50/20; A61B 5/0537; A61B 5/0816

(57) ABSTRACT

Systems and methods are disclosed. A device may include means for receiving physiologic information of a patient, including S4 heart sound information. A device may include an assessment means configured to: receive an indication of administered HCM therapy, determine a change in an HCM condition using the received S4 heart sound information by comparing S4 heart sound information over a first time period before the indication of administered HCM therapy with S4 heart sound information over a second time period subsequent to the indication of administered HCM therapy, provide an indication of HCM therapy efficacy to a user or process based on the determined change in HCM condition.

### CLAIM OF PRIORITY

This application claims the benefit of U.S. Provisional Application No. 63/555,636, filed on Feb. 20, 2024, and U.S. Provisional Application No. 63/565,440, filed on Mar. 14, 2024, each of which are hereby incorporated by reference in their entireties.

### TECHNICAL FIELD

This document relates generally to medical devices and more particularly to systems and methods to utilize S4 heart sounds to monitor hypertrophic cardiomyopathy (HCM) for a patient.

### BACKGROUND

Ambulatory medical devices (AMDs), including implantable, subcutaneous, wearable, or one or more other medical devices, etc., can monitor, detect, or treat various conditions, including heart failure (HF), atrial fibrillation (AF), etc. Ambulatory medical devices can include sensors to sense physiological information from a patient and one or more circuits to detect one or more physiologic events using the sensed physiological information or transmit sensed physiologic information or detected physiologic events to one or more remote devices. Frequent patient monitoring can provide early detection of worsening patient condition, including worsening heart failure or atrial fibrillation.

An arrhythmia is an abnormal heart rhythm (e.g., fast, slow, irregular, etc.). Arrhythmias include, among others, bradycardia, tachycardia, premature, extra, or skipped heart beats, and atrial or ventricular fibrillation affecting one or more chambers of the heart. Atrial fibrillation (AF) is as an abnormal heart rhythm characterized by rapid and irregular activity in the left or right atria of the heart. Atrial fibrillation is commonly associated with a reduction in cardiac output, an increased risk of heart failure, dementia, and stroke. Risk factors for atrial fibrillation include, among others, high blood pressure, heart failure, valvular heart disease, chronic obstructive pulmonary disorder (COPD), obesity, and sleep apnea.

Hypertrophic cardiomyopathy (HCM) is a condition in which muscle tissues of the heart become thickened. The most common affected part of the heart is the intraventricular septum, although other parts of the heart may also be affected. Treatments for the condition are possible, and include medications, ambulatory medical devices, and surgical intervention.

Ambulatory medical devices (AMDs), including implantable, subcutaneous, wearable, or one or more other medical devices, etc., can monitor, detect, or treat various conditions, including heart failure, atrial fibrillation, etc. Ambulatory medical devices can include sensors to sense physiological information from a patient and one or more circuits to detect one or more physiologic events using the sensed physiological information or transmit sensed physiologic information or detected physiologic events to one or more remote devices. Frequent patient monitoring can provide early detection of worsening patient condition, including worsening heart failure or atrial fibrillation.

Accurate identification of patients or groups of patients at an elevated risk of future adverse events may control mode or feature selection or resource management of one or more ambulatory medical devices, control notifications or messages in connected systems to various users associated with a specific patient or group of patients, organize or schedule physician or patient contact or treatment, or prevent or reduce patient hospitalization. Correctly identifying and safely managing patient risk of worsening condition may avoid unnecessary medical interventions, extend the usable life of ambulatory medical devices, and reduce healthcare costs.

### SUMMARY

Systems and methods are disclosed to determine and record one or more key metrics of heart sounds for a patient, including determining S4 heart sound information.

Example includes a medical device system. The system includes means for receiving physiologic information of a patient, including S4 heart sound information. The system also includes an assessment means configured to: receive an indication of administered HCM therapy; determine a change in an HCM condition using the received S4 heart sound information by comparing S4 heart sound information over a first time period before the indication of administered HCM therapy with S4 heart sound information over a second time period subsequent to the indication of administered HCM therapy; provide an indication of HCM therapy efficacy to a user or process based on the determined change in HCM condition.

Example 2 includes the medical device system of example 1, wherein to determine the change in the HCM condition includes to detect an increasing S4 heart sound to indicate an improving patient condition.

Example 3 includes the medical device system of example 1, wherein the means for receiving physiologic information of the patient further includes means for receiving S3 heart sound information; and wherein the assessment means is further configured to compare the received S3 heart sound information over the first time period before the indication of administered HCM therapy with S3 heart sound information over the second time period subsequent to the indication of administered HCM therapy.

Example 4 includes the medical device system of example 1, wherein to determine the change in the HCM condition includes to detect an increasing S4 heart sound and a stable or decreasing S3 heart sound to indicate an improving patient condition.

Example 5 includes the medical device system of example 1, wherein the means for receiving physiologic information of the patient further includes means for receiving S1 heart sound information; and wherein the assessment means is further configured to compare the received S1 heart sound information over the first time period before the indication of administered HCM therapy with S1 heart sound information over the second time period subsequent to the indication of administered HCM therapy.

Example 6 includes the medical device system of example 1, wherein to determine the change in the HCM condition includes to detect an increasing S4 heart sound and a stable or increasing S1 heart sound to indicate an improving patient condition.

Example 7 includes the medical device system of example 1, wherein the indication of administered HCM therapy includes an indication of septal ablation therapy.

Example 8 includes the medical device system of example 1, wherein the indication of administered HCM therapy includes an indication of septal myectomy therapy.

Example 9 includes the medical device system of any one of examples 1-6, wherein the indication of administered HCM therapy includes an indication of a pharmaceutical therapy.

Example 10 includes the medical device system of any one of examples 1-6, wherein the indication of administered HCM therapy includes an indication of exercise therapy.

Example 11 includes the medical device system of example 10, wherein the assessment means is configured to adjust the exercise therapy based on the determined change in the HCM condition and to provide the adjusted exercise therapy to the patient or a user or process.

Example 12 includes the medical device system of example 11, wherein the assessment means is configured to receive an indication of respiration, and to provide the adjusted exercise therapy to the patient or a user or process based on the determined change in the HCM condition and the indication of respiration.

Example 13 includes the medical device system of example 12, wherein indication of respiration includes respiration rate (RR) or rapid shallow breathing index (RSBI).

Example 14 includes the medical device system of any one of examples 1-6, wherein the means for receiving physiologic information is configured to receive impedance information of the patient, wherein the assessment means is configured to determine an indication of dehydration of the patient using the received impedance information and the received indication of administered HCM therapy and to provide the determined indication of dehydration to the patient or a user or process.

Example 15 includes the medical device system of any one of examples 1-6, wherein the assessment means is configured to provide a change in sample rate based on the change in the HCM condition.

Example 16 includes a medical device system the system includes a signal receiver circuit configured to receive physiologic information of a patient, including S4 heart sound information. The system also includes an assessment circuit configured to: to receive an indication of administered HCM therapy; determine a change in an HCM condition using the received S4 heart sound information by comparing S4 heart sound information over a first time period before the indication of administered HCM therapy with S4 heart sound information over a second time period subsequent to the indication of administered HCM therapy; provide an indication of HCM therapy efficacy to a user or process based on the determined change in HCM condition.

Example 17 includes the medical device system of example 16, wherein to determine the change in the HCM condition includes to detect an increasing S4 heart sound to indicate an improving patient condition.

Example 18 includes the medical device system of example 16, wherein the signal receiver circuit further includes means for receiving S3 heart sound information; and wherein the assessment circuit is further configured to compare the received S3 heart sound information over the first time period before the indication of administered HCM therapy with S3 heart sound information over the second time period subsequent to the indication of administered HCM therapy.

Example 19 includes the medical device system of example 16, wherein to determine the change in the HCM condition includes to detect an increasing S4 heart sound and a stable or decreasing S3 heart sound to indicate an improving patient condition.

Example 20 includes the medical device system of example 16, wherein the signal receiver circuit further includes means for receiving S1 heart sound information; and wherein the assessment circuit is further configured to compare the received S1 heart sound information over the first time period before the indication of administered HCM therapy with S1 heart sound information over the second time period subsequent to the indication of administered HCM therapy.

Example 21. The medical device system of example 16, wherein to determine the change in the HCM condition includes to detect an increasing S4 heart sound and a stable or increasing S1 heart sound to indicate an improving patient condition.

Example 22 includes the medical device system of example 16, wherein the indication of administered HCM therapy includes an indication of septal ablation therapy.

Example 23 includes the medical device system of example 16, wherein the indication of administered HCM therapy includes an indication of septal myectomy therapy.

Example 24 includes the medical device system of example 16, wherein the indication of administered HCM therapy includes an indication of a pharmaceutical therapy.

Example 25 includes the medical device system of example 16, wherein the indication of administered HCM therapy includes an indication of exercise therapy.

Example 26 includes the medical device system of example 25, wherein the assessment circuit is configured to adjust the exercise therapy based on the determined change in the HCM condition and to provide the adjusted exercise therapy to the patient or a user or process.

Example 27 includes the medical device system of example 26, wherein the assessment circuit is configured to receive an indication of respiration, and to provide the adjusted exercise therapy to the patient or a user or process based on the determined change in the HCM condition and the indication of respiration.

Example 28 includes the medical device system of example 27, wherein indication of respiration includes respiration rate (RR) or rapid shallow breathing index (RSBI).

Example 29 includes the medical device system of example 16, wherein the signal receiver circuit is configured to receive impedance information of the patient, wherein the assessment circuit is configured to determine an indication of dehydration of the patient using the received impedance information and the received indication of administered HCM therapy and to provide the determined indication of dehydration to the patient or a user or process.

Example 30 includes the medical device system of example 16, wherein the assessment circuit is configured to provide a change in sample rate based on the change in the HCM condition.

Example 31 includes a method. The method includes receiving physiologic information of a patient, including S4 heart sound information. The method also includes determining, using an assessment circuit: an indication of HCM for the patient; a change in an HCM condition using the received S4 heart sound information by comparing S4 heart sound information over a first time period before the indication of administered HCM therapy with S4 heart sound information over a second time period subsequent to the indication of administered HCM therapy; and determining, using the assessment circuit, an indication of patient condition based on the determined change in the HCM condition; and providing, using the assessment circuit, the determined indication of patient condition to a user or process.

Example 32 includes the method of example 31, wherein to determining the change in the HCM condition includes to detect an increasing S4 heart sound to indicate an improving patient condition.

Example 33 includes the method of example 31, further including receiving S3 heart sound information; and further including comparing the received S3 heart sound information over the first time period before the indication of administered HCM therapy with S3 heart sound information over the second time period subsequent to the indication of administered HCM therapy.

Example 34 includes the method of example 31, further including receiving S1 heart sound information; and further including comparing the received S1 heart sound information over the first time period before the indication of administered HCM therapy with S1 heart sound information over the second time period subsequent to the indication of administered HCM therapy.

Example 35 includes the method of example 31, wherein determining the indication of HCM for the patient includes determining an indication of a pharmaceutical therapy.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example diagram of monitoring HCM using a determined patient parameter.

FIG. 2 illustrates an example medical device system.

FIG. 3 illustrates an example patient management system and portions of an environment in which the system may operate.

FIG. 4 illustrates an example implantable medical device (IMD) electrically coupled to a heart.

FIG. 5 illustrates an example machine upon which any one or more of the techniques discussed herein may perform.

### DETAILED DESCRIPTION

Medical devices can be implanted in a patient or otherwise positioned on or about the patient to monitor patient physiologic information, such as heart sound information, respiration information (e.g., respiration rate (RR), tidal volume (TV), rapid shallow breathing index (RSBI), etc.), impedance information (e.g., intrathoracic impedance (ITTI)), pressure information, cardiac electrical information (e.g., heart rate), physical activity information, or other physiologic information or one or more other physiologic parameters of the patient, or to provide electrical stimulation or one or more other therapies or treatments to optimize or control contractions of a heart of the patient. For example, the medical device can include one or more implantable medical devices (IMDs), such as cardiac resynchronization therapy (CRT) devices, etc., configured to receive cardiac electrical information from, and in certain examples, provide electrical stimulation to, one or more electrodes located within, on, or proximate to the heart, such as coupled to one or more leads and located in one or more chambers of the heart, within the vasculature of the heart near one or more chambers, or otherwise attached to or in contact with the heart.

Stimulation signals can be generated and provided one or more chambers of the heart (e.g., frequently two or more of the right ventricle (RV), the left ventricle (LV) (e.g., commonly through the cardiac vasculature), or the right atrium (RA), etc.) to improve cardiac function, such as improving coordination of contractions between different chambers of the heart (e.g., the right ventricle and the left ventricle, the right atrium and the right ventricle, etc.) or otherwise improving cardiac output or efficiency. A number of types of physiological information may be used to evaluate a patient and/or vary settings in a medical device as described. One type of physiological information includes heart sound information.

Heart sounds are recurring mechanical signals associated with cardiac vibrations or accelerations from blood flow through the heart or other cardiac movements with each cardiac cycle and can be separated and classified according to activity associated with such vibrations, accelerations, movements, pressure waves, or blood flow. Heart sounds include four major features: the first through the fourth heart sounds (S1 through S4, respectively). The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle during diastole. An abrupt halt of early diastolic filling can cause the third heart sound (S3). Vibrations due to atrial kick can cause the fourth heart sound (S4). Valve closures and blood movement and pressure changes in the heart can cause accelerations, vibrations, or movement of the cardiac walls that can be detected using an accelerometer or a microphone, providing an output referred to herein as cardiac acceleration information.

Respiration information can include, among other things, a respiratory rate (RR) of the patient, a tidal volume (TV) of the patient, a rapid shallow breathing index (RSBI) of the patient, or other respiratory information of the patient. The respiratory rate is a measure of a breathing rate of the patient, generally measured in breaths per minute. The tidal volume is an aggregate measure of respiration changes, such as detected using measured changes in thoracic impedance, etc. The RSBI is a measure of respiratory frequency relative tidal volume of the patient. The nHR is a measure of heart rate (HR) of the patient at night, either in relation to sensing patient sleep or using a preset or selectable time of day corresponding to patient sleep.

Physiologic metrics, as described herein, can include one or more different measures of rate, amplitude, energy, etc., of different physiologic information over one or more time periods, such as representative daily values, etc. For example, heart sound metrics can be determined for each heart sound (e.g., the first heart sound (S1) through the fourth heart sound (S4), etc.) and can include an indication of an amplitude or energy of a specific heart sound for a specific cardiac cycle, or a representation of a number of cardiac cycles of the patient over a specific time period. Daily metrics can be determined representative of an average daily value for the patient, either corresponding to a waking time or a 24-hour period, etc. Respiration metrics can include, among other things, a mean or median respiration rate, binned values of rates, and a representative value of specific rate bins, etc. Heart rate metrics can include an average nighttime heart rate, a minimum nighttime heart rate, etc.

The activity information can include an activity measurement of the patient, such as detected using an accelerometer, a posture sensor, a step counter, or one or more other activity sensors associated with an ambulatory medical device. The impedance information can include, among other things, thoracic impedance information of the patient, such as a measure of impedance across a thorax of the patient from one or more electrodes associated with the ambulatory medical device (e.g., one or more leads of an implantable medical device proximate a heart of the patient and a housing of the implantable medical device implanted subcutaneously at a thoracic location of the patient, one or more external leads on a body of the patient, etc.). In other examples, the impedance information can include one or more other impedance measurements associated with the thorax of the patient, or otherwise indicative of patient thoracic impedance.

The temperature information can include an internal patient temperature at an ambulatory medical device, such as implanted in the thorax of the patient, or one or more other temperature measurements made at a specific location on the patient, etc. The temperature information can be detected using a temperature sensor, such as one or more circuits or electronic components having an electrical characteristic that changes with temperature. The temperature sensor can include a sensing element located on, at, or within the ambulatory medical device configured to determine a temperature indicative of patient temperature at the location of the ambulatory medical device.

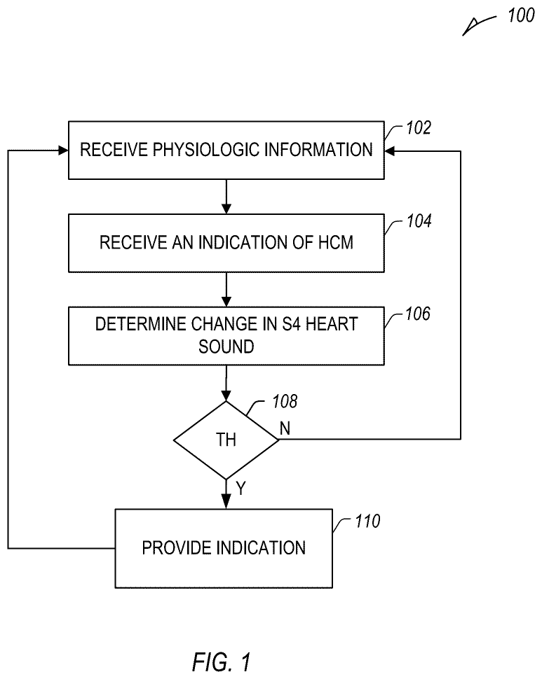

FIG. 1 illustrates an example process 100 to monitor HCM in a patient using one or more patient parameters that include an S4 heart sound. In operation 102, physiological information of a patient that includes S4 heart sound information is received. In selected examples, additional physiological information of a patient is also received along with S4 heart sound information. The additional physiological information may be used along with S4 heart sound information to further assess an HCM condition and provide additional indications to a user or process.

A number of sources of information to the process 100 are within the scope of the invention. In one example, receiving physiological information includes receiving from one or more biological sensors attached to, or adjacent to a patient. Examples include, but are not limited to, acoustic sensors to detect heart sounds such as the noted S4 heart sound and/or other heart sounds. Other examples include inductance sensors to measure an amount of hydration of a patient. Other examples include sensors to measure respiration. Other examples include sensors to measure physical activity, such as motion sensors, heart rate sensors, etc. In one example, physical activity sensors are correlated to physical activity program data as discussed below. Compliance with a physical activity program can be measured with data from physical activity sensors.

Sensors that are attached to, or adjacent to a patient may be included in one device, or multiple devices. The information collected may be processed in local assessment circuit included within the same device. The information collected may instead be transmitted remotely to a separate assessment circuit. Results from the assessment circuit may be transmitted back to a device attached to, or adjacent to a patient.

Selected information used by the assessment circuit may be included from sources that are not biological sensors. For example, an indication of HCM may come from a patient chart from a previous diagnosis. An indication of HCM may come from a physician and be directly input to an assessment circuit. In another example, an indication of a physical activity program may come from a patient chart, a physician, another heath specialist etc. The indication of a physical activity program may include only the existence of a physical activity program, or specific details of a physical activity program, such as suggested heart rate ranges, suggested respiration rate ranges, etc. In one example, indication of a physical activity program includes suggested rapid shallow breathing index (RSBI) information.

In one example, S4 heart sound information includes a presence or absence of an S4 heart sound. In one example, S4 heart sound information includes quantitative S4 heart sound information. One example of quantitative S4 heart sound information includes a magnitude of S4 heart sound. Other examples of quantitative S4 heart sound information include duration, sound variation, sound energy, vibration frequency, etc. In one example, an S4 heart sound is detected and/or quantified using vibration detection. Examples of vibration detection include, but are not limited to acoustic sensors or accelerometers. In one example, one or more quantitative S4 heart sound metrics are calculated from one or more components of measured instrument data, such as vibration magnitude as a function of time, etc. For example, S4 heart sound information may include an average S4 sound magnitude over a given time, a quantity of energy in a given window, etc.

In operation 104, an indication of HCM is received or detected, such as using one or more indications in a medical history of the patient, using received information from a clinician, etc. In known HCM patients, the inventors have recognized that S4 heart sound changes correlate with changes in HCM condition. In operation 106, a change in S4 heart sound information is determined over a first time period. In one example, the first time period includes a time period over multiple days (e.g., greater than 7 days, greater than 14 days, etc.), or a representative value (e.g., a daily value, a measure representative of a first number of daily values, such as 3 days, etc.) over a baseline representative of a longer time period (e.g., 14 days, 30 days, 90 days, etc.) Changes in chronic HCM will manifest over time periods of days, months, years, etc. In one example, the first time period is chosen to detect gradual changes of evolution of a chronic condition, in contrast to abrupt changes in a patient condition that require emergency response. In one example, the first time period is shorter than multiple days. In operation 106, the change in S4 heart sound information is correlated to a change in an HCM condition.

In one example, in operation 104, an indication of HCM that is received includes an indication of administered HCM therapy. After an administered HCM therapy it is desired to assess an effectiveness of the administered therapy. In examples discussed below, an effectiveness of administered HCM therapy is evaluated using an S4 heart sound. Additional physiological information may also be used in conjunction with an S4 heart sound to further assess an effectiveness of the administered HCM therapy. Examples of administered HCM therapy include, but are not limited to: an indication of septal ablation therapy; an indication of septal myectomy therapy; an indication of a pharmaceutical therapy (e.g., a myosin inhibitor, etc.); an indication of activities of daily living (ADL) therapy, such as exercise therapy, diet and nutrition therapy, personal behavior modification including stress management or a limitation in alcohol consumption or smoking; etc.

In certain examples, application or administration of a myosin inhibitor (e.g., mavacamten, etc.) for treatment of HCM can require echocardiogram assessment before treatment (e.g., to establish baseline cardiac function and confirm HCM diagnosis) and monitoring at regular intervals (e.g., at or before first application, 4 weeks, 8 weeks, 12 weeks, then every 12 weeks during treatment, etc.) to adjust dosage and monitor for complications. In an example, separate from or in addition echocardiogram sessions or appointments, the present inventors have recognized that S4 heart sound information can be used to monitor patient status during treatment, to adjust dosage, to adjust follow-up schedules, or to provide one or more alerts of changes in patient status associated with treatment or occurring coincident therewith.

As discussed in examples above, in one example a change in S4 heart sound that increases indicates a changing of an HCM condition. In one example a change in S4 heart sound that decreases indicates a change of an HCM condition. In one example a change in S4 heart sound indicates an improvement in HCM condition. In one example a change in S4 heart sound indicates a worsening in HCM condition. In one example an increase in S4 heart sound information includes an increase in maximum measured S4 magnitude. In one example an increase in S4 heart sound information includes an increase in an average measured S4 over time. Other changes in S4 heart sound that correlate to changing HCM condition include changes in variability of S4 heart sound over time. Other changes in S4 heart sound that correlate to changing HCM condition include a presence of an S4 heart sound at a first time, and an absence of an S4 heart sound at a second time later than the first time.

One of ordinary skill in the art, having the benefit of the present disclosure will recognize that a presence or absence of an S4 heart sound will be determined by a detectability threshold of data collection devices such as accelerometer data. In one example, detecting an absence of an S4 heart sound includes detecting an S4 sound for one or more cardiac cycles above an S4 loss threshold, and a subsequent value of the received S4 heart sound information for one or more cardiac cycles below the S4 loss threshold within the first time period.

In one example, an increasing S4 heart sound after treatment indicates improving patient condition and effective treatment. In certain examples, as an improvement to use of S4 heart sounds alone to determine patient condition or treatment efficacy, measures of S4 heart sounds can be combined with measures of S3 heart sounds, S1 heart sounds, or combinations thereof to determine patient condition or treatment efficacy.

In one example, adding detected S3 heart sound data in addition to S4 heart sound data provides enhanced characterization of a patient condition, such as improving, worsening or stabilizing, etc. In one example, an increasing S4 heart sound in combination with a stable S3 heart sound or a decreasing S3 heart sound indicates improving patient condition, and effective treatment. In an example, patient condition or treatment efficacy can be determined as a function of a measure of the S4 heart sound relative to a measure of the S3 heart sound (e.g., S4/S3, etc.).

In one example, adding detected S1 heart sound data in addition to S4 heart sound data provides enhanced characterization of a patient condition, such as improving, worsening or stabilizing, etc. In one example, an increasing S4 heart sound in combination with a stable S1 heart sound or an increasing S1 heart sound indicates improving patient condition, and effective treatment. In an example, patient condition or treatment efficacy can be determined as a function of a measure of the S4 heart sound and a measure of the S1 heart sound (e.g., S4*S1, etc.). In other examples, combinations of S1 and S3 heart sounds can be used to determine patient condition or treatment efficacy, such as a measure of S3 relative to S1 (e.g., S4*S1/S3, etc.).

In one example, an initial period after treatment leads to a decrease in S4 that coincides with a decrease in S1. In this example, if S4 flattens and then starts to increase after an initial decrease, this can indicate an unwanted side effect to treatment.

In operation 108, received data and/or a calculated S4 metric is applied to a threshold value or condition. If the threshold value or condition is exceeded, in operation 110 an indication is provided to a user or process based on the determined change in the HCM condition. An indication to a user or process may include a message that an HCM condition has changed. An indication to a user or process may include a message to a physician that an HCM condition has changed. An indication to a user or process may include an indication to a device to change device settings. An indication to a user or process may include an alert that an HCM condition is worsening.

In one example, an indication to a user or process may include an indication of efficacy of an HCM treatment previously administered. In one example, an indication to a user or process may include an indication that an additional HCM treatment is recommended. A severity of HCM may indicate different therapy treatment. For example, if a selected pharmaceutical treatment is warranted, the indication to a user or process may indicate that the pharmaceutical treatment threshold has been met. In one example, an indication to a user or process may include an indication that a hypertrophic septal ablation treatment is warranted. One example of a septal ablation treatment includes an alcohol septal ablation (ASA). In one example, an indication to a user or process may include an indication that a septal myectomy treatment is warranted. Although pharmaceutical treatment, septal ablation, and septal myectomy are used as examples, other surgical or non-surgical treatments are also within the scope of the invention. In other examples, an indication to a user or process may include a change to a device.

In one example, an indication to a user or process may include an indication of a diagnosis. In one example, a left ventricular outflow tract (LVOT) obstruction is detectable as a function of S4 heart sound changes. Because both HCM and a left ventricular outflow tract (LVOT) obstruction both indicate with an S4 heart sound, a left ventricular outflow tract (LVOT) obstruction can be difficult to distinguish. S4 heart sounds associated with a left ventricular outflow tract (LVOT) obstruction are dynamic in patients with HCM. The S4 heart sound increases with strain. One example of strain that can be used in diagnosis includes a Valsalva maneuver. Another similar strain includes bowel movements in a patient. In one example, physiological information of a patient received may include indication of strain, such as indication of a bowel movement. S4 heart sounds can then be monitored during a condition of strain (bowel movement, Valsalva maneuver, etc.) and an increase in S4 heart sound indicates a left ventricular outflow tract (LVOT) obstruction.

In one example, an indication to a user or process may include a change in data sampling rate for a device. In one example, a sample rate of data is increased if the S4 heart sound information indicates a worsening of an HCM condition. In one example, a sample rate of data is decreased if the S4 heart sound information indicates an improvement of an HCM condition.

In one example, receiving physiological information includes receiving an indication of physical activity over a time period. In operation 106, a change in S4 heart sound information is determined over a first time period. In operation 108, received data and/or a calculated S4 metric is applied to a threshold value or condition. If the threshold value or condition is exceeded, in operation 110 an indication is provided to a user or process based on the determined change in the HCM condition. In operation 110, the indication uses both the indication of physical activity and the calculated S4 metric to provide an indication of a recommended change in physical activity.

In one example, an indication of a recommended change in physical activity includes an alert to reduce physical activity. In one example, an indication of a recommended change in physical activity includes a recommended level of physical activity. In one example, an indication of a recommended change in physical activity includes a recommended change from a first physical activity plan to a second adjusted physical activity plan. Compliance with a physical activity plan may be monitored by a number of metrics, including, but not limited to respiration rate (RR), rapid shallow breathing index (RSBI), or other metrics.

In one example, in operation 102, the physiological information includes an indication of impedance information of the patient. Impedance information provides data that is correlated to a level of hydration or dehydration of a patient. In one example, impedance information is used in conjunction with physical activity to provide an indication of a recommended change in physical activity. In other examples, impedance information is used on its own, without physical activity information. In one example, the process 100 includes receiving an indication of a paradoxical split in S2 heart sound over the time period. A split S2 heart sound occurs when closure of the aortic valve and closure of the pulmonary valve are not synchronized, and is generally detected during inhalation. A paradoxical split S2 refers to splitting of S2 detected during exhalation rather than inhalation.

The information of a paradoxical split in S2 heart sound can be provided in a number of different ways. In one example, the process 100 includes detecting a paradoxical split in S2 heart sound over the time period. Detecting involves a sensor that receives data such as heart sound data and respiration data. The received data can be used to determine a paradoxical split in S2 heart sound. In one example, the process 100 includes receiving an indication of a paradoxical split in S2 heart sound over the time period. Receiving may include getting a recorded instance of a paradoxical split in S2 heart sound from a patient's medical history.

A paradoxical split in S2 heart sound can indicate a need for aortic valve replacement. In one example, aortic valve replacement includes transcatheter aortic valve replacement (TAVR), although the invention is not so limited. The inventors have recognized that in patients with aortic valve disease, an S4 sound may also be present. By monitoring for both a paradoxical split in S2 and changes in S4 heart sound, it can be determined whether a patient needs treatment for HCM or if the patient more urgently needs aortic valve replacement. In operation 110, the indication uses both the indication of paradoxical split in S2 heart sound and the calculated S4 metric to provide an indication of a recommended aortic valve replacement.

In other examples, separate from S4, or in combination therewith, determination of a change in HCM condition or an indication of HCM therapy efficacy can more broadly be based on physiologic monitoring following a received indication of administered HCM therapy, including administration of a myosin inhibitor or one or more other HCM therapies.

For example, application of a myosin inhibitor may result in a reduction in cardiac contractility. A reduction in cardiac contractility can result in a reduced ejection fraction (EF). In certain examples, determined indications or measures of heart failure can change based on changes in cardiac contractility or ejection fraction. For example, a decrease in ejection fraction may result in an increase in a determined measure of heart failure.

In an example, separate from S4, a detected increase in S3 can result in an increase in a determined measure of heart failure, in certain examples indicating a reduction in ejection of blood from the ventricles. The present inventors have recognized, among other things, separate measures of myosin inhibitor efficacy can be determined using different measures. For example, myosin inhibitor efficacy can be determined as a function of determined measures of S1 of a patient, indicating a force of ventricular contraction. Changes in determined measures of S1 with application of HCM therapy, including administration of a myosin inhibitor, can provide an indication of patient response to the applied HCM therapy. Separately, determined measures of S3 can indicate a patient ability to tolerate the dosage of applied HCM therapy. In certain examples, a combined measure, such as a measure of S3 over S1, or a detected increase in S3 for a given reduction in S1, can indicate patient response and dosage optimization, with the reduction in S1 indicative of HCM therapy efficacy and the increase in S3 below a threshold indicative of dosage optimization. In certain examples, each of the S1 and S3 measures can be compared to separate first and second thresholds. In other examples, the composite measure of S3 over S1 can be compared to a third threshold, in certain examples in combination with one or more of the first and second thresholds for S1 and S3.

In an example, a detected change in S1 below a threshold can indicate that the patient is not a responder to the applied HCM therapy, and in response, an alert can be provided that HCM therapy can be discontinued or a higher dosage can be applied. In another example, a detected change in S3 above at threshold can indicate a decrease or worsened patient condition or an increase in a determined measure of patient heart failure. In certain examples, the S1 and S3 thresholds can depend on each other, such that a greater increase in S3 can be tolerated without alert with a corresponding decrease in S1, where the greater increase can, in certain examples, be based on the determined decrease in S1. However, an increase in S3 without a decrease in S1 can trigger an alert to cease the applied therapy.

An example function for an HCM therapy efficacy measure can be represented as follows:

$$( a * Δ S 3 ) / ( b * Δ S 1 ) \qquad ( 1 )$$

In the HCM therapy efficacy measure above, therapy can continue or increase until the HCM therapy efficacy measure exceeds a threshold. In other examples, separate measures for S1 or S3 can be implemented having separate thresholds. For example, therapy can continue or increase until an increase in S3 measure exceeds a threshold. In another example, therapy can increase until a decrease in S1 measure exceeds a threshold. In contrast, if therapy is increased to a maximum level without a corresponding decrease in the S1 measure, therapy can be discontinued, and the patient can be determined as a non-responder. In other examples, combinations of the different measures can be implemented in various permutations.

Ambulatory monitoring of HCM patients corresponding to HCM therapy, and in certain examples prior to HCM therapy to establish a baseline before therapy (e.g., from which delta measurements can be determined) can be advantageous over regular, in-clinic follow-up echocardiogram monitoring at regular intervals. For example, patient progress and condition can be constantly monitored, reducing the time to determine optimal HCM therapy dosage. Instead of a follow-up at 4 weeks to determine patient response to HCM therapy, real-time response and stabilization can be determined and detected, enabling remote adjustment of HCM therapy or to optimize echocardiogram or clinician follow-up schedules or timing. For example, stabilization of patient condition, such as determined as a function of S3, S1, or combinations thereof, can be detected after initial HCM therapy or following a change in dosage or administration, enabling remote clinician review of patient condition and determination of one or more changes in HMC therapy. In addition, a decrease in patient condition can be determined as it occurs, and not merely at one or more scheduled follow-up appointments (e.g., after 4 or 12 weeks, etc.).

In other examples, one or more other determinations of left ventricular function separate from (or in combination with) S1 or S3 can be determined coincident with HCM therapy or HCM therapy changes or adjustments. For example, electrocardiogram signals can be detected by an ambulatory medical device and analyzed, such as at the ambulatory medical device or using one or more remote devices, to determine changes in left ventricular function. For example, a machine or deep learning model (e.g., a deep neural network, etc.) can be developed based on training data to analyze electrocardiogram signals (and in certain examples echocardiogram signals) and determine, based on features in the signals or signal changes over time, such as coincident with HCM therapy, left ventricular function or changes in left ventricular function, such as left ventricular ejection fraction (LVEF), etc. Early emergence of heart failure or increases in determined heart failure measures can be determined based on application of device-detected electrocardiogram signals applied to one or more deep learning models, in certain examples in combination with heart sound measures (e.g., S1, S3, etc.), to cease or adjust HCM therapy, such as changing an HCM therapy dosage, periodizing recovery periods between therapy sessions (e.g., to prevent adaptation or allow patient recovery, etc.).

In other examples, one or more composite measures of patient condition can be used to determine patient response or to adjust HCM therapy or follow-up scheduling. One example of a composite indication is a HeartLogic™ index, a HeartLogic™ in-alert time, or one or more other composite measurements or measures thereof. The HeartLogic™ index is a composite indication of patient condition determined using different combinations or weightings of physiologic information, including two or more of S1 heart sounds, S3 heart sounds, thoracic impedance, activity information, respiration information, and nighttime heart rate (nHR). The HeartLogic™ index can be indicative of a heart failure status, a risk a heart failure event (e.g., within in a given time period), or a worsening of the heart failure status or risk of heart failure event in the patient over time. The HeartLogic™ in-alert time is a measure of time that the HeartLogic™ index is above an alert threshold.

In certain examples, the different combinations or weightings of physiologic information used to determine the HeartLogic™ index can be adjusted or determined based on a risk stratifier. In certain examples, the risk stratifier can be determined as a different combination of physiologic information, including one or more of S3, respiratory rate, and time active (e.g., an amount of time at a specific activity level above a mean activity level of the patient or a specific threshold, etc.). For example, if the risk stratifier is low, or below a first threshold, the HeartLogic™ index can be determined using a first combination of physiologic information. If the risk stratifier is high, or above a second threshold, the HeartLogic™ index can be determined using a second combination of physiologic information, such as additional information than included in the first combination (e.g., the first combination and the second combination, etc.). If the risk stratifier is between the first and second thresholds, the HeartLogic™ index can be determined using the first combination and one or more metrics or components of the second combination, or using the first combination and the second combination, but with the second combination having less weight than if the risk stratifier is above the second threshold (e.g., using less of the second combination than the first combination).

In an example, the HeartLogic™ index and in-alert time can include worsening heart failure or physiologic event detection, including risk indication or stratification, such as that disclosed in the commonly assigned An et al. U.S. Pat. No. 9,968,266 entitled “RISK STRATIFICATION BASED HEART FAILURE DETECTION ALGORITHM,” or in the commonly assigned An et al. U.S. Pat. No. 9,622,664 entitled “METHODS AND APPARATUS FOR DETECTING HEART FAILURE DECOMPENSATION EVENT AND STRATIFYING THE RISK OF THE SAME,” or in the commonly assigned Thakur et al. U.S. Pat. No. 10,660,577 entitled “SYSTEMS AND METHODS FOR DETECTING WORSENING HEART FAILURE,” or in the commonly assigned An et al. U.S. Patent Application No. 2014/0031643 entitled “HEART FAILURE PATIENT STRATIFICATION,” or in the commonly assigned Thakur et al. U.S. Pat. No. 10,085,696 entitled “DETECTION OF WORSENING HEART FAILURE EVENTS USING HEART SOUNDS,” each of which are hereby incorporated by reference in their entireties, including their disclosures of heart failure and worsening heart failure detection, heart failure risk indication detection, and stratification of the same, etc.

In an example, a detected increase in a determined value of the HeartLogic™ index can indicate worsening patient condition. A detected increase or determined value above a threshold, such as in response to HCM therapy or a change in HCM therapy, can indicate a worsened patient condition. Responsive to a detected worsened patient condition above a threshold, an alert can be provided to reduce or cease HCM therapy, or one or more processes can adjust HCM therapy or instructions for HCM therapy based on the value of the HeartLogic™ index. In certain examples, a detected increase in a determined value of the HeartLogic™ index below a threshold, indicating that the patient condition is stable or relatively stable, can be used to support or increase HCM therapy, such as to increase a dosage or rate of dosage increase, etc.

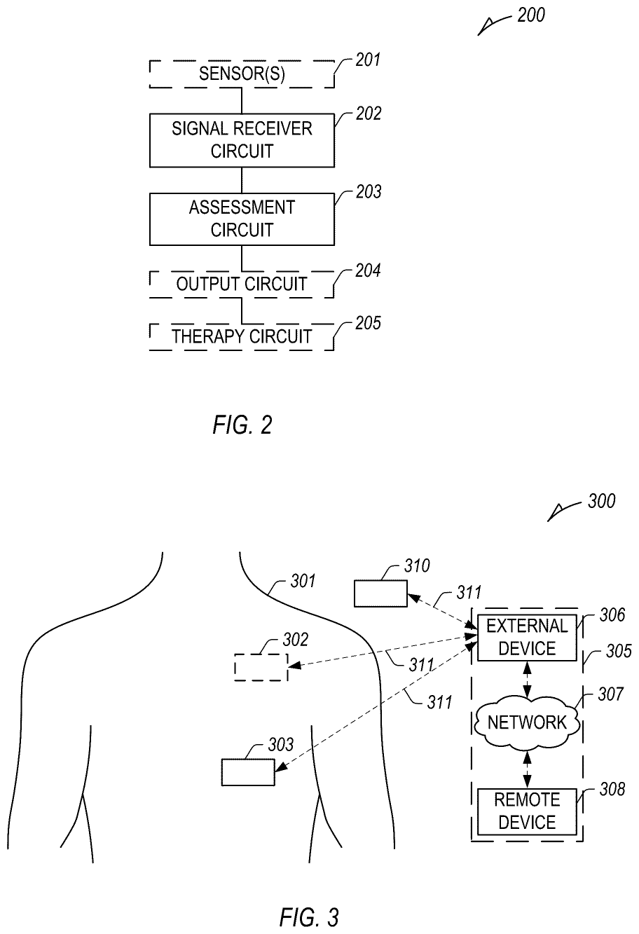

FIG. 2 illustrates an example system 200 (e.g., a medical device system). In an example, one or more aspects of the example system 200 can be a component of, or communicatively coupled to, a medical device, such as an implantable medical device (IMD), an insertable cardiac monitor (ICM), an ambulatory medical device (AMD), etc. The system 200 can be configured to monitor, detect, or treat various physiologic conditions of the body, such as cardiac conditions associated with a reduced ability of a heart to sufficiently deliver blood to a body, including heart failure, arrhythmias, dyssynchrony, etc., or one or more other physiologic conditions and, in certain examples, can be configured to provide electrical stimulation or one or more other therapies or treatments to the patient.

The system 200 can include a single medical device or a plurality of medical devices implanted in a patient's body or otherwise positioned on or about the patient to monitor patient physiologic information of the patient using information from one or more sensors, such as a sensor 201. In an example, the sensor 201 can include one or more of: a respiration sensor configured to receive respiration information (e.g., a respiratory rate, a respiration volume (tidal volume), etc.); an acceleration sensor (e.g., an accelerometer, a microphone, etc.) configured to receive cardiac acceleration information (e.g., cardiac vibration information, pressure waveform information, heart sound information, endocardial acceleration information, acceleration information, activity information, posture information, etc.); an impedance sensor (e.g., an intrathoracic impedance sensor, a transthoracic impedance sensor, a thoracic impedance sensor, etc.) configured to receive impedance information; a cardiac sensor configured to receive cardiac electrical information; an activity sensor configured to receive information about a physical motion (e.g., activity, steps, etc.); a posture sensor configured to receive posture or position information; a pressure sensor configured to receive pressure information; a plethysmograph sensor (e.g., a photoplethysmography sensor, etc.); a chemical sensor (e.g., an electrolyte sensor, a pH sensor, an anion gap sensor, etc.); a temperature sensor; a skin elasticity sensor, or one or more other sensors configured to receive physiologic information of the patient.

The example system 200 can include a signal receiver circuit 202 and an assessment circuit 203. The signal receiver circuit 202 can be configured to receive physiologic information of a patient (or group of patients) from the sensor 201. The assessment circuit 203 can be configured to receive information from the signal receiver circuit 202, and to determine one or more parameters (e.g., physiologic parameters, stratifiers, etc.) or existing or changed patient conditions (e.g., indications of patient dehydration, respiratory condition, cardiac condition (e.g., heart failure, arrhythmia), sleep disordered breathing, etc.) using the received physiologic information, such as described herein. The physiologic information can include, among other things, cardiac electrical information, impedance information, respiration information, heart sound information, activity information, posture information, temperature information, or one or more other types of physiologic information.

In certain examples, the assessment circuit 203 can aggregate information from multiple sensors or devices, detect various events using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

In certain examples, such as to detect an improved or worsening patient condition, some initial assessment is often required to establish a baseline level or condition from one or more sensors or physiologic information. Subsequent detection of a deviation from the baseline level or condition can be used to determine the improved or worsening patient condition. However, in other examples, the amount of variation or change (e.g., relative or absolute change) in physiologic information over different time periods can used to determine a risk of an adverse medical event, or to predict or stratify the risk of the patient experiencing an adverse medical event (e.g., a heart failure event) in a period following the detected change, in combination with or separate from any baseline level or condition.

Changes in different physiologic information can be aggregated and weighted based on one or more patient-specific stratifiers and, in certain examples, compared to one or more thresholds, for example, having a clinical sensitivity and specificity across a target population with respect to a specific condition (e.g., heart failure), etc., and one or more specific time periods, such as daily values, short term averages (e.g., daily values aggregated over a number of days), long term averages (e.g., daily values aggregated over a number of short term periods or a greater number of days (sometimes different (e.g., non-overlapping) days than used for the short term average)), etc.

The system 200 can include an output circuit 204 configured to provide an output to a user, or to cause an output to be provided to a user, such as through an output, a display, or one or more other user interface, the output including a score, a trend, an alert, or other indication. In other examples, the output circuit 204 can be configured to provide an output to another circuit, machine, or process, such as a therapy circuit 205 (e.g., a cardiac resynchronization therapy (CRT) circuit, a chemical therapy circuit, a stimulation circuit, etc.), etc., to control, adjust, or cease a therapy of a medical device, a drug delivery system, etc., or otherwise alter one or more processes or functions of one or more other aspects of a medical device system, such as one or more CRT parameters, drug delivery, dosage determinations or recommendations, etc. In an example, the therapy circuit 205 can include one or more of a stimulation control circuit, a cardiac stimulation circuit, a neural stimulation circuit, a dosage determination or control circuit, etc. In other examples, the therapy circuit 205 can be controlled by the assessment circuit 203, or one or more other circuits, etc. In certain examples, the assessment circuit 203 can include the output circuit 204 or can be configured to determine the output to be provided by the output circuit 204, while the output circuit 204 can provide the signals that cause the user interface to provide the output to the user based on the output determined by the assessment circuit 203.

A technological problem exists in medical devices and medical device systems that in low-power monitoring modes, ambulatory medical devices powered by one or more rechargeable or non-rechargeable batteries (e.g., including IMDs) have to make certain tradeoffs between battery life, or in the instance of implantable medical devices with non-rechargeable batteries, between device replacement periods often including surgical procedures, and sampling resolution, sampling periods, of processing, storage, and transmission of sensed physiologic information, or features or mode selection of or within the medical devices. Medical devices can include higher-power modes and lower-power modes. Physiologic information, such as indicative of a potential adverse physiologic event, can be used to transition from a low-power mode to a high-power mode. In certain examples, the low-power mode can include a low resource mode, characterized as requiring less power, processing time, memory, or communication time or bandwidth (e.g., transferring less data, etc.) than a corresponding high-power mode. The high-power mode can include a relatively higher resource mode, characterized as requiring more power, processing time, memory, or communication time or bandwidth than the corresponding low-power mode. However, by the time physiologic information detected in the low-power mode indicates a possible event, valuable information has been lost, unable to be recorded in the high-power mode.

The inverse is also true, in that false or inaccurate determinations that trigger a high-power mode unnecessarily unduly limit the usable life of certain ambulatory medical devices. For numerous reasons, it is advantageous to accurately detect and determine physiologic events, and to avoid unnecessary transitions from the low-power mode to the high-power mode to improve use of medical device resources.

For example, a change in modes can enable higher resolution sampling or an increase in the sampling frequency or number or types of sensors used to sense physiologic information leading up to and including a potential event. For example, different physiologic information is often sensed using non-overlapping time periods of the same sensor, in certain examples, at different sampling frequencies and power costs. In one example, heart sounds and patient activity can be detected using non-overlapping time periods of the same, single- or multi-axis accelerometer, at different sampling frequencies and power costs. In certain examples, a transition to a high-power mode can include using the accelerometer to detect heart sounds throughout the high-power mode, or at a larger percentage of the high-power mode than during a corresponding low-power mode, etc. In other examples, waveforms for medical events can be recorded, stored in long-term memory, and transferred to a remote device for clinician review. In certain examples, only a notification that an event has been stored is transferred, or summary information about the event. In response, the full event can be requested for subsequent transmission and review. However, even in the situation where the event is stored and not transmitted, resources for storing and processing the event are still by the medical device.

FIG. 3 illustrates an example patient management system 300 and portions of an environment in which the patient management system 300 may operate. The patient management system 300 can perform a range of activities, including remote patient monitoring and diagnosis of a disease condition. Such activities can be performed proximal to a patient 301, such as in a patient home or office, through a centralized server, such as in a hospital, clinic, or physician office, or through a remote workstation, such as a secure wireless mobile computing device.

The patient management system 300 can include one or more medical devices, an external system 305, and a communication link 311 providing for communication between the one or more ambulatory medical devices and the external system 305. The one or more medical devices can include an ambulatory medical device (AMD), such as an implantable medical device (IMD) 302, a wearable medical device 303, or one or more other implantable, leadless, subcutaneous, external, wearable, or medical devices configured to monitor, sense, or detect information from, determine physiologic information about, or provide one or more therapies to treat various conditions of the patient 301, such as one or more cardiac or non-cardiac conditions (e.g., dehydration, sleep disordered breathing, etc.).

In an example, the implantable medical device 302 can include one or more cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 301. In another example, the implantable medical device 302 can include a monitor implanted, for example, subcutaneously in the chest of patient 301, the implantable medical device 302 including a housing containing circuitry and, in certain examples, one or more sensors, such as a temperature sensor, etc.

Cardiac rhythm management devices, such as insertable cardiac monitors (ICMs), pacemakers, defibrillators, or cardiac resynchronizers, include implantable or subcutaneous devices having hermetically sealed housings configured to be implanted in a chest of a patient. The cardiac rhythm management device can include one or more leads to position one or more electrodes or other sensors at various locations in or near the heart, such as in one or more of the atria or ventricles of a heart, etc. Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured to detect physiologic information from the patient, or provide one or more therapies or stimulation to the patient.

Implantable devices can additionally or separately include leadless cardiac pacemakers (LCPs), small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemakers can be implanted in or about the heart to detect physiologic information from, or provide one or more therapies or stimulation to, one or more chambers of the heart. The multiple leadless cardiac pacemaker can communicate between themselves, or one or more other implanted or external devices.

The implantable medical device 302 can include an assessment circuit configured to detect or determine specific physiologic information of the patient 301, or to determine one or more conditions or provide information or an alert to a user, such as the patient 301 (e.g., a patient), a clinician, or one or more other caregivers or processes, such as described herein. The implantable medical device 302 can alternatively or additionally be configured as a therapeutic device configured to treat one or more medical conditions of the patient 301. The therapy can be delivered to the patient 301 via the lead system and associated electrodes or using one or more other delivery mechanisms. The therapy can include delivery of one or more drugs to the patient 301, such as using the implantable medical device 302 or one or more of the other ambulatory medical devices, etc. In some examples, therapy can include CRT for rectifying dyssynchrony and improving cardiac function in heart failure patients. In other examples, the implantable medical device 302 can include a drug delivery system, such as a drug infusion pump to deliver drugs to the patient for managing arrhythmias or complications from arrhythmias, hypertension, hypotension, or one or more other physiologic conditions. In other examples, the implantable medical device 302 can include one or more electrodes configured to stimulate the nervous system of the patient or to provide stimulation to the muscles of the patient airway, etc.

The wearable medical device 303 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 305 can include a dedicated hardware/software system, such as a programmer, a remote server-based patient management system, or alternatively a system defined predominantly by software running on a standard personal computer. The external system 305 can manage the patient 301 through the implantable medical device 302 or one or more other ambulatory medical devices connected to the external system 305 via a communication link 311. In other examples, the implantable medical device 302 can be connected to the wearable medical device 303, or the wearable medical device 303 can be connected to the external system 305, via the communication link 311. This can include, for example, programming the implantable medical device 302 to perform one or more of acquiring physiologic data, performing at least one self-diagnostic test (such as for a device operational status), analyzing the physiologic data, or optionally delivering or adjusting a therapy for the patient 301. Additionally, the external system 305 can send information to, or receive information from, the implantable medical device 302 or the wearable medical device 303 via the communication link 311. Examples of the information can include real-time or stored physiologic data from the patient 301, diagnostic data, such as detection of patient hydration status, hospitalizations, responses to therapies delivered to the patient 301, or device operational status of the implantable medical device 302 or the wearable medical device 303 (e.g., battery status, lead impedance, etc.). The communication link 311 can be an inductive telemetry link, a capacitive telemetry link, or a radio-frequency (RF) telemetry link, or wireless telemetry based on, for example, “strong” Bluetooth or IEEE 802.11 wireless fidelity “Wi-Fi” interfacing standards. Other configurations and combinations of patient data source interfacing are possible.

The external system 305 can include an external device 306 in proximity of the one or more ambulatory medical devices, and a remote device 308 in a location relatively distant from the one or more ambulatory medical devices, in communication with the external device 306 via a communication network 307. Examples of the external device 306 can include a medical device programmer. The remote device 308 can be configured to evaluate collected patient information and provide alert notifications, among other possible functions. In an example, the remote device 308 can include a centralized server acting as a central hub for collected data storage and analysis from a number of different sources. Combinations of information from the multiple sources can be used to make determinations and update individual patient status or to adjust one or more alerts or determinations for one or more other patients. The server can be configured as a uni-, multi-, or distributed computing and processing system. The remote device 308 can receive data from multiple patients. The data can be collected by the one or more ambulatory medical devices, among other data acquisition sensors or devices associated with the patient 301. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text, or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

The remote device 308 may additionally include one or more locally configured clients or remote clients securely connected over the communication network 307 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 308, including the server and the interconnected clients, may also execute a follow-up scheme by sending follow-up requests to the one or more ambulatory medical devices, or by sending a message or other communication to the patient 301 (e.g., the patient), clinician or authorized third party as a compliance notification.

The communication network 307 can provide wired or wireless interconnectivity. In an example, the communication network 307 can be based on the Transmission Control Protocol/Internet Protocol (TCP/IP) network communication specification, although other types or combinations of networking implementations are possible. Similarly, other network topologies and arrangements are possible.

One or more of the external device 306 or the remote device 308 can output the detected medical events to a system user, such as the patient or a clinician, or to a process including, for example, an instance of a computer program executable in a microprocessor. In an example, the process can include an automated generation of recommendations for anti-arrhythmic therapy, or a recommendation for further diagnostic test or treatment. In an example, the external device 306 or the remote device 308 can include a respective display unit for displaying the physiologic or functional signals, or alerts, alarms, emergency calls, or other forms of warnings to signal the detection of arrhythmias. In some examples, the external system 305 can include an external data processor configured to analyze the physiologic or functional signals received by the one or more ambulatory medical devices, and to confirm or reject the detection of arrhythmias. Computationally intensive algorithms, such as machine-learning algorithms, can be implemented in the external data processor to process the data retrospectively to detect cardiac arrhythmias.

Portions of the one or more ambulatory medical devices or the external system 305 can be implemented using hardware, software, firmware, or combinations thereof. Portions of the one or more ambulatory medical devices or the external system 305 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

The therapy device 310 can be configured to send information to or receive information from one or more of the ambulatory medical devices or the external system 305 using the communication link 311. In an example, the one or more ambulatory medical devices, the external device 306, or the remote device 308 can be configured to control one or more parameters of the therapy device 310. The external system 305 can allow for programming the one or more ambulatory medical devices and can receives information about one or more signals acquired by the one or more ambulatory medical devices, such as can be received via a communication link 311. The external system 305 can include a local external implantable medical device programmer. The external system 305 can include a remote patient management system that can monitor patient status or adjust one or more therapies such as from a remote location.

In certain examples, event storage can be triggered, such as received physiologic information or in response to one or more detected events or determined parameters meeting or exceeding a threshold (e.g., a static threshold, a dynamic threshold, or one or more other thresholds based on patient or population information, etc.). Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected atrial fibrillation event can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, amount of memory, etc.). Storing multiple windows using this early detection leading up to a single event can provide full event assessment with power and cost savings, in contrast to the longer loop recorder windows. In addition, early detection can trigger additional parameter computation or storage, at different resolution or sampling frequency, without unduly taxing finite system resources.

In certain examples, one or more alerts can be provided, such as to the patient, to a clinician, or to one or more other caregivers (e.g., using a patient smart watch, a cellular or smart phone, a computer, etc.), such as in response to the transition to the high-power mode, in response to the detected event or condition, or after updating or transmitting information from a first device to a remote device. In other examples, the medical device itself can provide an audible or tactile alert to warn the patient of the detected condition. For example, the patient can be alerted in response to a detected condition so they can engage in corrective action, such as sitting down, etc.

In certain examples, a therapy can be provided in response to the detected condition. For example, a pacing therapy can be provided, enabled, or adjusted, such as to disrupt or reduce the impact of the detected atrial fibrillation event. In other examples, delivery of one or more drugs (e.g., a vasoconstrictor, pressor drugs, etc.) can be triggered, provided, or adjusted, such as using a drug pump, in response to the detected condition, alone or in combination with a pacing therapy, such as that described above, such as to increase arterial pressure, maintain cardiac output, and to disrupt or reduce the impact of the detected atrial fibrillation event.

In certain examples, physiologic information of a patient can be sensed, such as by one or more sensors located within, on, or proximate to the patient, such as a cardiac sensor, a heart sound sensor, or one or more other sensors described herein. For example, cardiac electrical information of the patient can be sensed using a cardiac sensor. In other examples, cardiac acceleration information of the patient can be sensed using a heart sound sensor. The cardiac sensor and the heart sound sensor can be components of one or more (e.g., the same or different) medical devices (e.g., an implantable medical device, an ambulatory medical device, etc.). Timing metrics between different features (e.g., first and second cardiac features, etc.) can be determined, such as by a processing circuit of the cardiac sensor or one or more other medical devices or medical device components, etc. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc.

Heart sounds are recurring mechanical signals associated with cardiac vibrations or accelerations from blood flow through the heart or other cardiac movements with each cardiac cycle or interval and can be separated and classified according to activity associated with such vibrations, accelerations, movements, pressure waves, or blood flow. Heart sounds include four major features: the first through the fourth heart sounds (S1 through S4, respectively). The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle during diastole. An abrupt halt of early diastolic filling can cause the third heart sound (S3). Vibrations due to atrial kick can cause the fourth heart sound (S4). Valve closures and blood movement and pressure changes in the heart can cause accelerations, vibrations, or movement of the cardiac walls that can be detected using an accelerometer or a microphone, providing an output referred to herein as cardiac acceleration information.

In an example, heart sound signal portions, or values of respective heart sound signals for a cardiac interval, can be detected as amplitudes occurring with respect to one or more cardiac electrical features or one or more energy values with respect to a window of the heart sound signal, often determined with respect to one or more cardiac electrical features. For example, the value and timing of an S1 signal can be detected using an amplitude or energy of the heart sound signal occurring at or about the R wave of the cardiac interval. An S4 signal portion can be determined, such as by a processing circuit of the heart sound sensor or one or more other medical devices or medical device components, etc. In certain examples, the S4 signal portion can include a filtered signal from an S4 window of a cardiac interval. In an example, the S4 interval can be determined as a set time period in the cardiac interval with respect to one or more other cardiac electrical or mechanical features, such as forward from one or more of the R wave, the T wave, or one or more features of a heart sound waveform, such as the first, second, or third heart sounds (S1, S2, S3), or backwards from a subsequent R wave or a detected S1 of a subsequent cardiac interval. In certain examples, the length of the S4 window can depend on heart rate or one or more other factors. In an example, the timing metric of the cardiac electrical information can be a timing metric of a first cardiac interval, and the S4 signal portion can be an S4 signal portion of the same first cardiac interval.

In an example, a heart sound parameter can include information of or about multiple of the same heart sound parameter or different combinations of heart sound parameters over one or more cardiac cycles or a specified time period (e.g., 1 minute, 1 hour, 1 day, 1 week, etc.). For example, a heart sound parameter can include a composite S1 parameter representative of a plurality of S1 parameters, for example, over a certain time period (e.g., a number of cardiac cycles, a representative time period, etc.).

In an example, the heart sound parameter can include an ensemble average of a particular heart sound over a heart sound waveform, such as that disclosed in the commonly assigned Siejko et al. U.S. Pat. No. 7,115,096 entitled “THIRD HEART SOUND ACTIVITY INDEX FOR HEART FAILURE MONITORING,” or in the commonly assigned Patangay et al. U.S. Pat. No. 7,853,327 entitled “HEART SOUND TRACKING SYSTEM AND METHOD,” each of which are hereby incorporated by reference in their entireties, including their disclosures of ensemble averaging an acoustic signal and determining a particular heart sound of a heart sound waveform. In other examples, the signal receiver circuit can receive the at least one heart sound parameter or composite parameter, such as from a heart sound sensor or a heart sound sensor circuit.

In an example, cardiac electrical information of the patient can be received, such as using a signal receiver circuit of a medical device, from a cardiac sensor (e.g., one or more electrodes, etc.) or cardiac sensor circuit (e.g., including one or more amplifier or filter circuits, etc.). In an example, the received cardiac electrical information can include the timing metric between the first and second cardiac features of the patient.

In an example, cardiac acceleration information of the patient can be received, such as using the same or different signal receiver circuit of the medical device, from a heart sound sensor (e.g., an accelerometer, etc.) or heart sound sensor circuit (e.g., including one or more amplifier or filter circuits, etc.). In an example, the received cardiac acceleration information can include the S4 signal portion occurring between the first and second cardiac features of the patient. In certain examples, additional physiologic information can be received, such as one or more of heart rate information, activity information of the patient, or posture information of the patient, from one or more other sensor or sensor circuits.

In certain examples, a high-power mode can be in contrast to a low-power mode, and can include one or more of: enabling one or more additional sensors, transitioning from a low-power sensor or set of sensors to a higher-power sensor or set of sensors, triggering additional sensing from one or more additional sensors or medical devices, increasing a sensing frequency or a sensing or storage resolution, increasing an amount of data to be collected, communicated (e.g., from a first medical device to a second medical device, etc.), or stored, triggering storage of currently available information from a loop recorder in long-term storage or increasing the storage capacity or time period of a loop recorder, or otherwise altering device behavior to capture additional or higher-resolution physiologic information or perform more processing, etc.

Additionally, or alternatively, event storage can be triggered. Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected atrial fibrillation event can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, etc.).

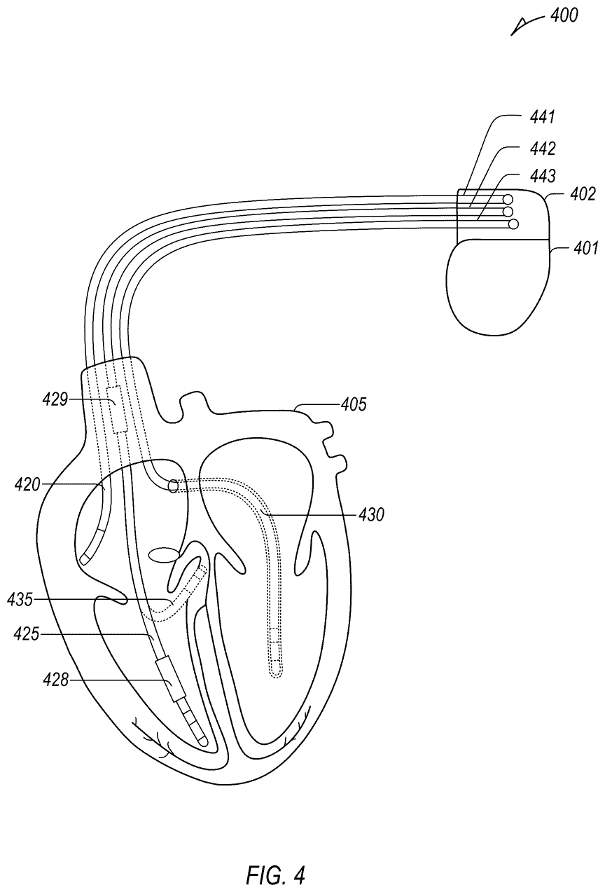

FIG. 4 illustrates an example implantable medical device (IMD) 400 electrically coupled to a heart 405, such as through one or more leads coupled to the implantable medical device 400 through one or more lead ports, such as first, second, or third lead ports 441, 442, 443 in a header 402 of the implantable medical device 400. In an example, the implantable medical device 400 can include an antenna, such as in the header 402, configured to enable communication with an external system (e.g., the external system 305) and one or more electronic circuits (e.g., the assessment circuit 203) in a hermetically sealed housing (CAN) 401.

The implantable medical device 400 may include an implantable cardiac monitor (ICM), pacemaker, defibrillator, cardiac resynchronizer, or other subcutaneous implantable medical device or cardiac rhythm management (CRM) device configured to be implanted in a chest of a subject, having one or more leads to position one or more electrodes or other sensors at various locations in or near the heart 405, such as in one or more of the atria or ventricles. Separate from, or in addition to, the one or more electrodes or other sensors of the leads, the implantable medical device 400 can include one or more electrodes or other sensors (e.g., a pressure sensor, an accelerometer, a gyroscope, a microphone, etc.) powered by a power source in the implantable medical device 400. The one or more electrodes or other sensors of the leads, the implantable medical device 400, or a combination thereof, can be configured detect physiologic information from, or provide one or more therapies or stimulation to, the patient.

Implantable devices can additionally include a leadless cardiac pacemaker (LCP), small (e.g., smaller than traditional implantable devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart 405 without traditional lead or implantable device complications (e.g., required incision and pocket, complications associated with lead placement, breakage, or migration, etc.). In certain examples, a leadless cardiac pacemaker can have more limited power and processing capabilities than a traditional CRM device; however, multiple leadless cardiac pacemaker devices can be implanted in or about the heart to detect physiologic information from, or provide one or more therapies or stimulation to, one or more chambers of the heart. The multiple LCP devices can communicate between themselves, or one or more other implanted or external devices.

The implantable medical device 400 can include one or more electronic circuits configured to sense one or more physiologic signals, such as an electrogram or a signal representing mechanical function of the heart 405. In certain examples, the CAN 401 may function as an electrode such as for sensing or pulse delivery. For example, an electrode from one or more of the leads may be used together with the CAN 401 such as for unipolar sensing of an electrogram or for delivering one or more pacing pulses. A defibrillation electrode (e.g., the first defibrillation coil electrode 428, the second defibrillation coil electrode 429, etc.) may be used together with the CAN 401 to deliver one or more cardioversion/defibrillation pulses.

In an example, the implantable medical device 400 can sense impedance such as between electrodes located on one or more of the leads or the CAN 401. The implantable medical device 400 can be configured to inject current between a pair of electrodes, sense the resultant voltage between the same or different pair of electrodes, and determine impedance, such as using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing, etc. In an example, the implantable medical device 400 can be configured to inject current between an electrode on one or more of the first, second, third, or fourth leads 420, 425, 430, 435 and the CAN 401, and to sense the resultant voltage between the same or different electrodes and the CAN 401.

The implantable medical device 400 can integrate one or more other physiologic sensors to sense one or more other physiologic signals, such as one or more of heart rate, heart rate variability, intrathoracic impedance, intracardiac impedance, arterial pressure, pulmonary artery pressure, RV pressure, LV coronary pressure, coronary blood temperature, blood oxygen saturation, one or more heart sounds, physical activity or exertion level, physiologic response to activity, posture, respiration, body weight, or body temperature. The arrangement and functions of these leads and electrodes are described above by way of example and not by way of limitation. Depending on the need of the patient and the capability of the implantable device, other arrangements and uses of these leads and electrodes are possible.

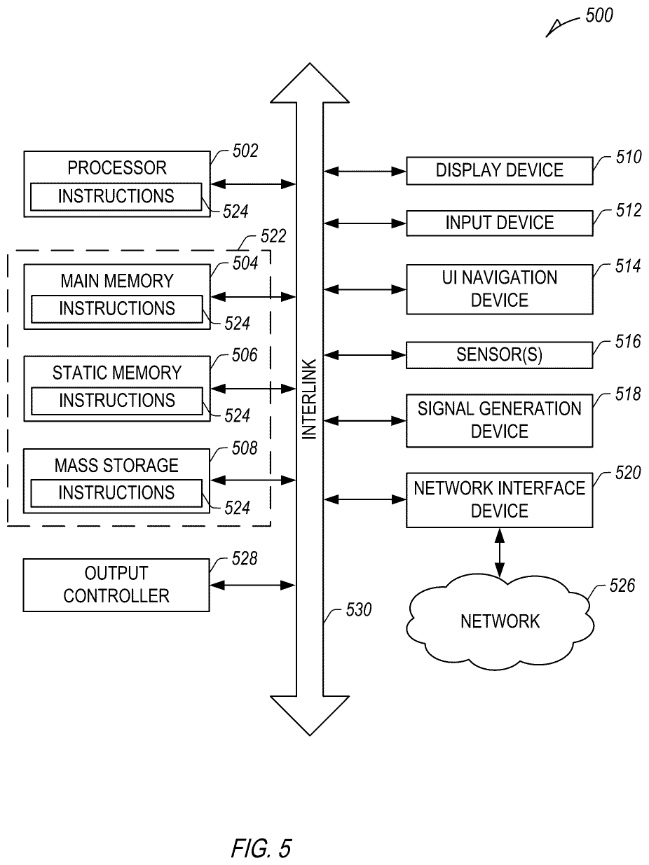

FIG. 5 illustrates a block diagram of an example machine 500 upon which any one or more of the techniques (e.g., methodologies) discussed herein may perform. Portions of this description may apply to the computing framework of one or more of the medical devices described herein, such as the implantable medical device, the external programmer, etc. Further, as described herein with respect to medical device components, systems, or machines, such may require regulatory-compliance not capable by generic computers, components, or machinery.

Examples, as described herein, may include, or may operate by, logic or a number of components, or mechanisms in the machine 500. Circuitry (e.g., processing circuitry, an assessment circuit, etc.) is a collection of circuits implemented in tangible entities of the machine 500 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 500 follow.

In alternative embodiments, the machine 500 may operate as a standalone device or may be connected (e.g., networked) to other machines. In a networked deployment, the machine 500 may operate in the capacity of a server machine, a client machine, or both in server-client network environments. In an example, the machine 500 may act as a peer machine in peer-to-peer (P2P) (or other distributed) network environment. The machine 500 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 500 (e.g., computer system) may include a hardware processor 502 (e.g., a central processing unit (CPU), a graphics processing unit (GPU), a hardware processor core, or any combination thereof), a main memory 504, a static memory 506 (e.g., memory or storage for firmware, microcode, a basic-input-output (BIOS), unified extensible firmware interface (UEFI), etc.), and mass storage 508 (e.g., hard drive, tape drive, flash storage, or other block devices) some or all of which may communicate with each other via an interlink 530 (e.g., bus). The machine 500 may further include a display unit 510, an input device 512 (e.g., a keyboard), and a user interface (UI) navigation device 514 (e.g., a mouse). In an example, the display unit 510, input device 512, and UI navigation device 514 may be a touch screen display. The machine 500 may additionally include a signal generation device 518 (e.g., a speaker), a network interface device 520, and one or more sensors 516, such as a global positioning system (GPS) sensor, compass, accelerometer, or one or more other sensors. The machine 500 may include an output controller 528, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the hardware processor 502, the main memory 504, the static memory 506, or the mass storage 508 may be, or include, a machine-readable medium 522 on which is stored one or more sets of data structures or instructions 524 (e.g., software) embodying or utilized by any one or more of the techniques or functions described herein. The instructions 524 may also reside, completely or at least partially, within any of registers of the hardware processor 502, the main memory 504, the static memory 506, or the mass storage 508 during execution thereof by the machine 500. In an example, one or any combination of the hardware processor 502, the main memory 504, the static memory 506, or the mass storage 508 may constitute the machine-readable medium 522. While the machine-readable medium 522 is illustrated as a single medium, the term “machine-readable medium” may include a single medium or multiple media (e.g., a centralized or distributed database, and/or associated caches and servers) configured to store the one or more instructions 524.

The term “machine-readable medium” may include any medium that is capable of storing, encoding, or carrying instructions for execution by the machine 500 and that cause the machine 500 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 524 may be further transmitted or received over a communications network 526 using a transmission medium via the network interface device 520 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 520 may include one or more physical jacks (e.g., Ethernet, coaxial, or phone jacks) or one or more antennas to connect to the communications network 526. In an example, the network interface device 520 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 500, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A medical device system, comprising:
a signal receiver circuit configured to receive physiologic information of a patient, including S4 heart sound information; and
an assessment circuit configured to:
to receive an indication of administered HCM therapy;
determine a change in an HCM condition using the received S4 heart sound information by comparing S4 heart sound information over a first time period before the indication of administered HCM therapy with S4 heart sound information over a second time period subsequent to the indication of administered HCM therapy;
provide an indication of HCM therapy efficacy to a user or process based on the determined change in HCM condition.

2. The medical device system of claim 1, wherein to determine the change in the HCM condition includes to detect an increasing S4 heart sound to indicate an improving patient condition.

3. The medical device system of claim 1, wherein the signal receiver circuit further includes means for receiving S3 heart sound information; and
wherein the assessment circuit is further configured to compare the received S3 heart sound information over the first time period before the indication of administered HCM therapy with S3 heart sound information over the second time period subsequent to the indication of administered HCM therapy.

4. The medical device system of claim 1, wherein to determine the change in the HCM condition includes to detect an increasing S4 heart sound and a stable or decreasing S3 heart sound to indicate an improving patient condition.

5. The medical device system of claim 1, wherein the signal receiver circuit further includes means for receiving S1 heart sound information; and
wherein the assessment circuit is further configured to compare the received S1 heart sound information over the first time period before the indication of administered HCM therapy with S1 heart sound information over the second time period subsequent to the indication of administered HCM therapy.

6. The medical device system of claim 1, wherein to determine the change in the HCM condition includes to detect an increasing S4 heart sound and a stable or increasing S1 heart sound to indicate an improving patient condition.

7. The medical device system of claim 1, wherein the indication of administered HCM therapy includes an indication of septal ablation therapy.

8. The medical device system of claim 1, wherein the indication of administered HCM therapy includes an indication of septal myectomy therapy.

9. The medical device system of claim 1, wherein the indication of administered HCM therapy includes an indication of a pharmaceutical therapy.

10. The medical device system of claim 1, wherein the indication of administered HCM therapy includes an indication of exercise therapy.

11. The medical device system of claim 10, wherein the assessment circuit is configured to adjust the exercise therapy based on the determined change in the HCM condition and to provide the adjusted exercise therapy to the patient or a user or process.

12. The medical device system of claim 11, wherein the assessment circuit is configured to receive an indication of respiration, and to provide the adjusted exercise therapy to the patient or a user or process based on the determined change in the HCM condition and the indication of respiration.

13. The medical device system of claim 12, wherein indication of respiration includes respiration rate (RR) or rapid shallow breathing index (RSBI).

14. The medical device system of claim 1, wherein the signal receiver circuit is configured to receive impedance information of the patient,
wherein the assessment circuit is configured to determine an indication of dehydration of the patient using the received impedance information and the received indication of administered HCM therapy and to provide the determined indication of dehydration to the patient or a user or process.

15. The medical device system of claim 1, wherein the assessment circuit is configured to provide a change in sample rate based on the change in the HCM condition.

16. A method, comprising:
receiving physiologic information of a patient, including S4 heart sound information; and
determining, using an assessment circuit:
an indication of HCM for the patient;
a change in an HCM condition using the received S4 heart sound information by comparing S4 heart sound information over a first time period before the indication of administered HCM therapy with S4 heart sound information over a second time period subsequent to the indication of administered HCM therapy; and
determining, using the assessment circuit, an indication of patient condition based on the determined change in the HCM condition; and
providing, using the assessment circuit, the determined indication of patient condition to a user or process.

17. The method of claim 16, wherein to determining the change in the HCM condition includes to detect an increasing S4 heart sound to indicate an improving patient condition.

18. The method of claim 16, further including receiving S3 heart sound information; and
further including comparing the received S3 heart sound information over the first time period before the indication of administered HCM therapy with S3 heart sound information over the second time period subsequent to the indication of administered HCM therapy.

19. The method of claim 16, further including receiving S1 heart sound information; and
further including comparing the received S1 heart sound information over the first time period before the indication of administered HCM therapy with S1 heart sound information over the second time period subsequent to the indication of administered HCM therapy.

20. The method of claim 16, wherein determining the indication of HCM for the patient includes determining an indication of a pharmaceutical therapy.
